(19) United States
(12) Patent Application Publication
(10) Pub. No.: US 2022/0265203 A1
(43) Pub. Date: Aug. 25, 2022

(54) Systems and Methods for Aiding Labor

(71) Applicant: Roshane Sasson, Hackensack, NJ (US)

(72) Inventor: Roshane Sasson, Hackensack, NJ (US)

(21) Appl. No.: 17/679,799

(22) Filed: Feb. 24, 2022

Related U.S. Application Data
(60) Provisional application No. 63/152,851, filed on Feb. 24, 2021.

(51) Int. Cl. A61B 5/00

(52) U.S. Cl. CPC A61B 5/4356; A61B 5/743; A61B 5/002; A61B 5/0024; A61B 5/7475; A61B 5/0011

(57) ABSTRACT

Methods and systems implementing a graphical user interface for making childbirth more comfortable and/or effective during labor are disclosed. In some embodiments, the systems and methods are configured to receive uterine pressure strength data and use an algorithm to implement a graphical user interface displaying the current uterine pressure, a patient-defined threshold, and/or a color panel indicating a comfort or contraction zone. In some embodiments, the graphical user interface displays, based on the zone, Montevideo units, a timer, and an estimated time until the end of the current zone in seconds. In some embodiments, the systems and methods calculate when minimal and low changes are being made to the uterine pressure, informing the graphical user interface to output information corresponding to the users' needs and/or wants. In some embodiments, chimes can be activated on a graphical user interface, indicating a transition from a comfort zone to a contraction zone.

### CROSS-REFERENCE TO RELATED APPLICATIONS

This application is related to and claims priority benefits from U.S. Provisional Application Ser. No. 63/152,851 filed on Feb. 24, 2021, entitled “Systems and Methods for Aiding Labor”.

The '851 application is hereby incorporated by reference herein in its entirety.

### FIELD OF THE INVENTION

This invention relates to methods, algorithms, systems, and computer applications for making labor more comfortable and/or effective. In some embodiments, the invention relates to methods, algorithms, systems, and/or computer applications that can show when a patient is having a uterine contraction, predict the time when future contractions will likely occur and/or end, and/or provide an updated Montevideo unit score depicting a patient's progression of labor. In some embodiments, this information can be displayed on a graphical user interface. In some embodiments, patient specified information can be displayed during childbirth.

Modern childbirth has evolved into a complicated process that often involves multiple medical monitors, trained healthcare providers, and/or the use of various pain-relieving drugs and/or epidurals. However, natural birth still depends on the woman pushing the baby through the birth canal. Epidurals and pain-relieving drugs used to bring patients comfort during the labor and delivery process can prevent a woman from realizing when she needs to push or when her contractions have arrived. This setback can lead to an augmented second stage of labor and cause dystocia (lack of progress in labor). The longer the second stage of labor lasts, the more discomfort patients experience, in addition to an increase in expenses, infections, perineal tears, and women undergoing Cesareans. Having adequate contraction pushes are pivotal in creating a successful vaginal delivery.

Many ways currently exist to provide and showcase information during the progression of labor, such as maternal monitors. Fetal monitors, in conjunction with intrauterine pressure catheters, are often used to monitor the uterine activity of women in labor, as well as the condition of the fetus in utero. However, the information provided by these tools is often only viewed by clinical staff and/or interpreted through a medium unfit for most patients in labor. Providing and explaining this technical information to expectant mothers during labor is often difficult for healthcare providers who are often understaffed.

Therefore, a need exists for methods and systems that generate and display information to patients that promote more comfortable and/or effective childbirths. In at least some embodiments, this can be accomplished by methods and systems that can predict and display when patients' future contractions will occur and/or end. In at least some embodiments, the systems and methods can offer comforting stimuli to the patients.

### SUMMARY OF THE INVENTION

Methods and systems for receiving, storing, and accessing data to be used by an application to comfort a patient during childbirth and/or make childbirth more efficient are disclosed below.

A system for making labor more comfortable and/or efficient can include a monitor configured to measure patient data; a computer; a display and/or a server. In some embodiments, the monitor monitors the uterine pressure strength of a patient. In some embodiments, the monitor is a fetal monitor. In some embodiments, the monitor is a maternal monitor.

In some embodiments, the patient data is analyzed by a computer to determine when a patient is having a contraction. In some embodiments, the patient data is analyzed by the computer to predict when a patient will have a contraction. In some embodiments, the patient data is analyzed by the computer to predict when a contraction will end. In some embodiments, the patient data is analyzed by the computer to predict how long a current and/or future contraction will last.

In some embodiments, the display is configured to show a graphical user interface. In some embodiments, the graphical user interface includes a Comfort Panel. In some embodiments, the Comfort Panel changes between colors whether a patient is experiencing a contraction.

In some embodiments, the graphical user interface includes a Montevideo score. In some embodiments, the Montevideo score is updated at regular intervals. In some embodiments, a patient can turn the Montevideo score on and off.

In some embodiments, the monitor is an intrauterine pressure catheter sensor.

In some embodiments, the system can output motivational messages on the display.

In some embodiments, the system is configured to offer a comforting stimulus. In some embodiments, the comforting stimulus is an audio sound, an image, a video, a scent, a taste, or a physical stimulation.

In some embodiments, a computer readable medium can have program code recorded thereon for execution on a computer of the methods disclosed herein.

Methods and systems for making labor more comfortable and/or efficient are disclosed below.

### BRIEF DESCRIPTION OF THE DRAWINGS

FIG. 1A illustrates a flow diagram of an embodiment of a communication module providing data via a server to a computer application implementing a graphical user interface to make childbirth more comfortable.

FIG. 1B illustrates a flow diagram of an embodiment of a communication module providing data directly to a device capable of displaying a graphical user interface to make childbirth more comfortable.

FIG. 1C illustrates a flow diagram of an embodiment of multiple communication modules providing data to a computer application implementing a graphical user interface that can be used in an institutional setting, such as a hospital.

FIG. 2A shows a flow diagram of a software algorithm executed by an application according to some embodiments.

FIG. 2B shows a flow diagram of another software algorithm executed by an application according to some embodiments.

FIG. 3A depicts an example graphical user interface for displaying user related uterine information for a patient in between contractions.

FIG. 3B depicts an example of a graphical user interface for displaying user related uterine information for a patient during a contraction.

FIG. 4 is an example of a graphical user interface for displaying user related uterine information for multiple patients.

FIG. 5A and 5B show a flow diagram of a software algorithm executed by an application according to some embodiments.

### DETAILED DESCRIPTION OF PREFERRED EMBODIMENT(S)

In some embodiments, methods and systems can be used to bring comfort to women during childbirth. In some embodiments, methods such as showing when a patient is having a contraction and predicting when their future contractions will begin and/or end through a graphical user interface are used. In some embodiments, the methods and systems receive fetal data and/or uterine pressure strength data collected from a fetal monitor and/or a maternal monitor and send the data to a server. In some embodiments, other devices that collect uterine pressure strength can be used. In some embodiments, a user can manually enter information such as uterine pressure strength and/or timing of contractions. In some embodiments, a server sends that data to a computer for processing. In some embodiments, the information received is processed via an algorithm. In some embodiments, the application can determine and inform the patient whether they are currently having a contraction based on fluctuations in intrauterine data. In some embodiments, the system provides patients with a time prediction of when their next uterine contraction will start. In some embodiments, a graphical user interface utilizes colors to represent whether the patient is having a contraction and/or is in a contraction state. In some embodiments, a scoring system is featured to inform the patient of their performance calculated using Montevideo units. In some embodiments, the Montevideo score is calculated by obtaining the peak uterine pressure amplitude and subtracting the resting tone over a given interval (such as a ten-minute window).

In some embodiments the Montevideo score can be continuously updated as contractions occur. In some embodiments, the Montevideo score is updated at a regular interval, for example every 10 minutes. In some embodiments, within an interval, if the patient has a score above and/or equal to a given number, such as 200, the system can indicate, for example by changing color, that the patient has surpassed the interval goal required to induce labor. In some embodiments, the indication lasts for the duration of the interval.

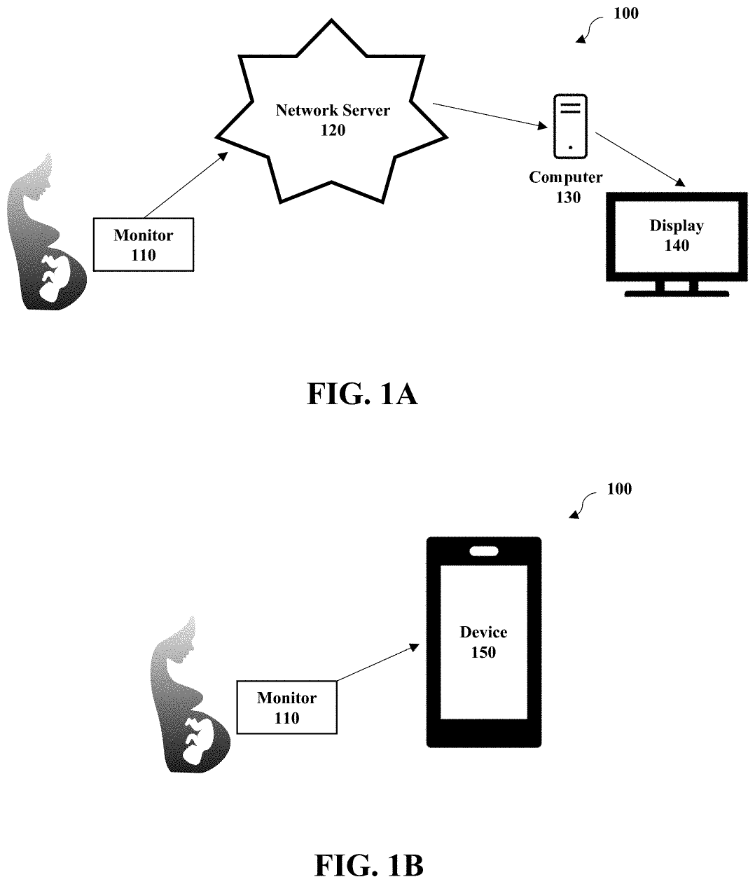

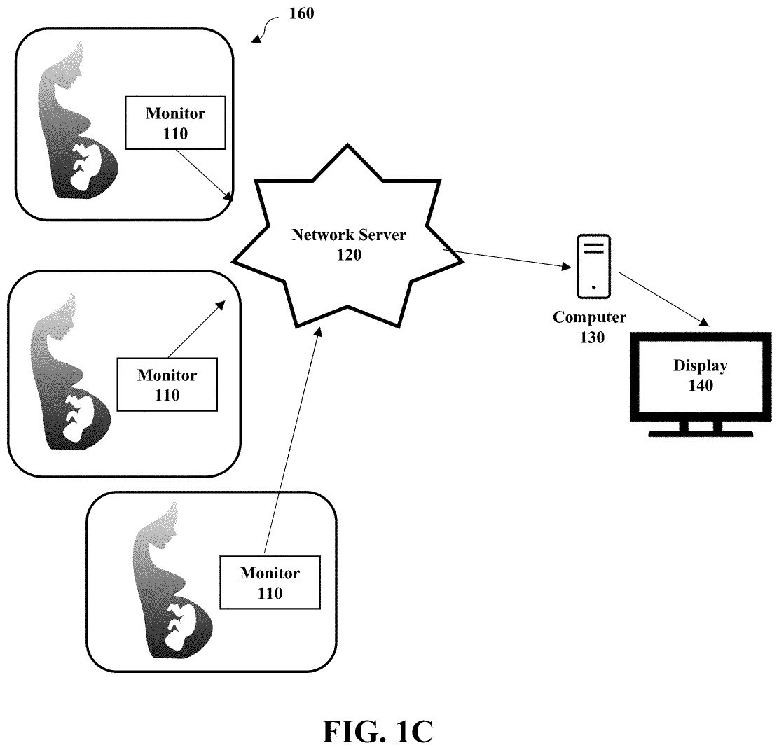

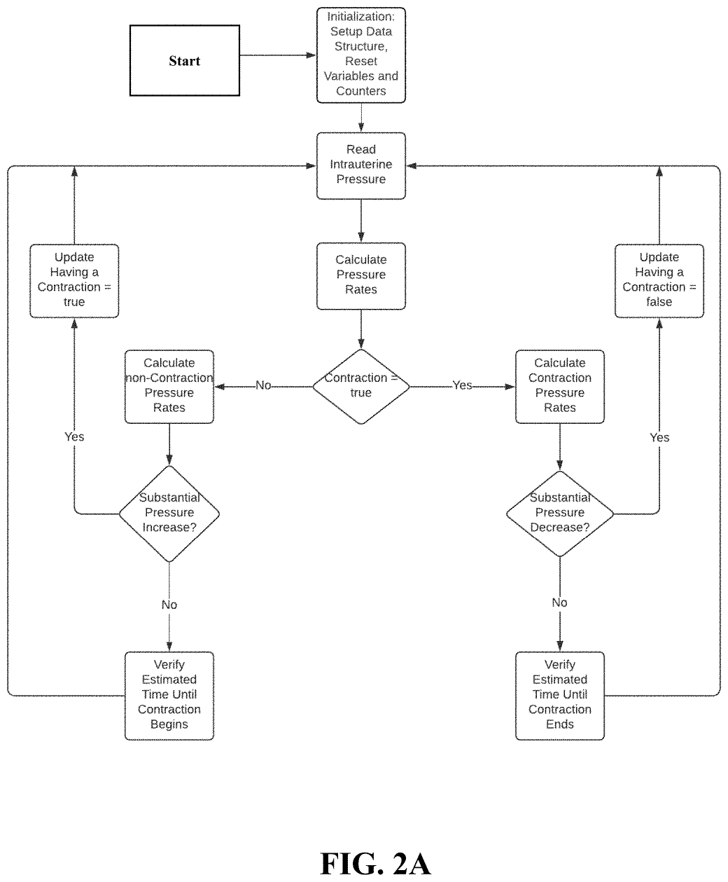

FIG. 1 illustrates an embodiment of real-time environment 100 comprising monitor 110, network server 120, computer 130 and display unit 140.

In some embodiments, monitor 110 is a maternal monitor. In some embodiments, monitor 110 is a uterine activity monitor. In some embodiments, monitor 110 tracks uterine and/or fetal activity. In some embodiments, an intrauterine pressure catheter can be used to measure uterine intensity and strength. In some embodiments, providing comprehensive perinatal monitoring allows for access to detailed maternal information. In some embodiments, the access is essentially immediate.

In some embodiments, monitor 110 includes an IUPC (intrauterine pressure catheter) sensor used within the monitoring system. In some embodiments, the IUPC can be replaced with a tocodynamometer and/or an electrohysterograph. In some embodiments, other types of sensors can be configured to communicate with network server 120 such as, but not limited to, uterine activity monitors. In some embodiments, monitor 110 is a device that can capture and transmit uterine strength data. In some embodiments, this data can be streamed to a network. In some embodiments, this network can be accessed by multi-chat servers.

In some embodiments, monitor 110 is directly connected to a network. In some embodiments, monitor 110 can transmit signals via one of several wireless communications protocols, such as Bluetooth, WiFi, CDMA, 900 MHz, 3G/4G/5G/Cellular, near-field communication, and/or other communication protocols to a network.

In some embodiments, network server 120 receives data from monitor 110 and transmits it to computer 130. In some embodiments, monitor 110 transmits data directly to computer 130 which then displays said data on display unit 140. In some embodiments, such as the one depicted in FIG. 1B, monitor 110 transmits data directly to device 150. In some embodiments, monitor 110 transmits data directly to device 150 via a Bluetooth signal.

In some embodiments, network server 120 receives uterine pressure strength data from monitoring system 110 and sends it to computer 130 for processing. In some embodiments, monitoring system 110 and computer 130 each respectively participate as clients to network server 120. In some embodiments, monitoring system 110 only sends information to network server 120 and computer 130 only reads information from network server 120.

In some embodiments, computer 130 connects to network server 120 via an IP address and port number in order to receive data. In some embodiments, network server 120 communicates as a multi-chat server. In some embodiments, as monitoring system 110 receives the data and streams it to network server 120, network server 120 sends the data for processing. In some embodiments, network server 120 accepts various forms of data streaming

In some embodiments, computer 130 is one of a smartphone, a tablet, or a computer, such as a single-board computer. In some embodiments, computer 130 is not capable of displaying information. In some embodiments, computer 130 and display unit 140 are the same. In some embodiments, display unit 140 is one of a smartphone, a tablet, or a personal computer. In some embodiments, device 150 is one of a smartphone, a tablet, or a personal computer.

In at least some embodiments, display unit 140 and/or device 150 display a graphical user interface.

In some embodiments, a plurality of perspective environments 160 exist simultaneously. In some embodiments, such as depicted in FIG. 1C, a separate system can be setup with display windows showing a live feed of each perspective environment 160 within the plurality. Doctors and nurses can utilize this to monitor multiple patients and assess their overall comfortability and/or progress.

In some embodiments, computer 130 can be replaced with other devices such as laptops, smartphones, desktop computers and tablets. In some embodiments, display unit 140 acts as computer 130 and both processes the data sent from monitor 110 and displays the results on a graphical user interface.

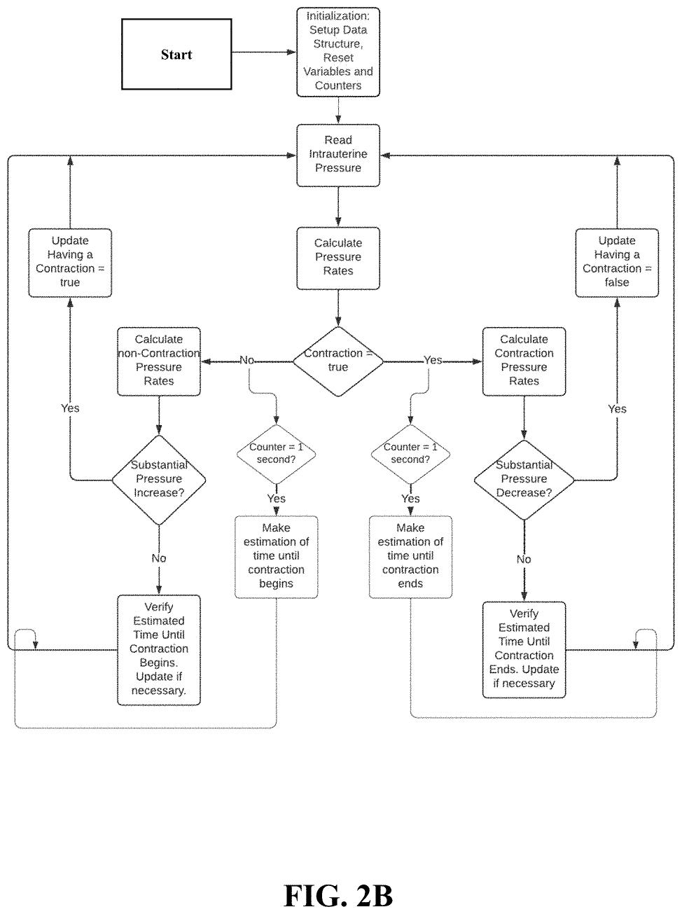

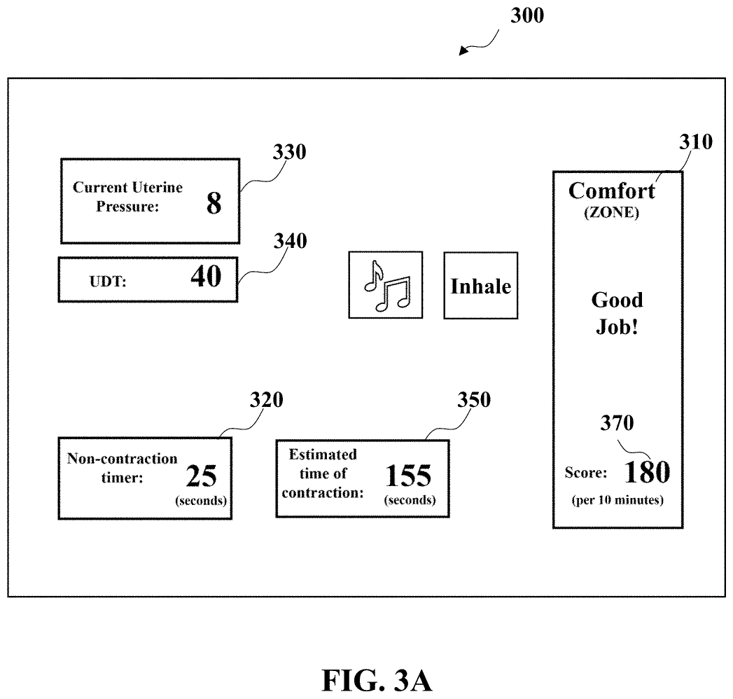

FIG. 2A shows a flow diagram according to some embodiments in which a computer reads uterine pressure data and indicates whether a patient is having a contraction. In some embodiments, uterine pressure rates are gathered periodically to assess the possibility of a contraction occurring. In at least some embodiments, based on the results, predictions are made to estimate when the beginning and/or ending of a given contraction will start and/or end. In some embodiments, the patient can customize how early contraction detections occur with a specified pressure threshold.

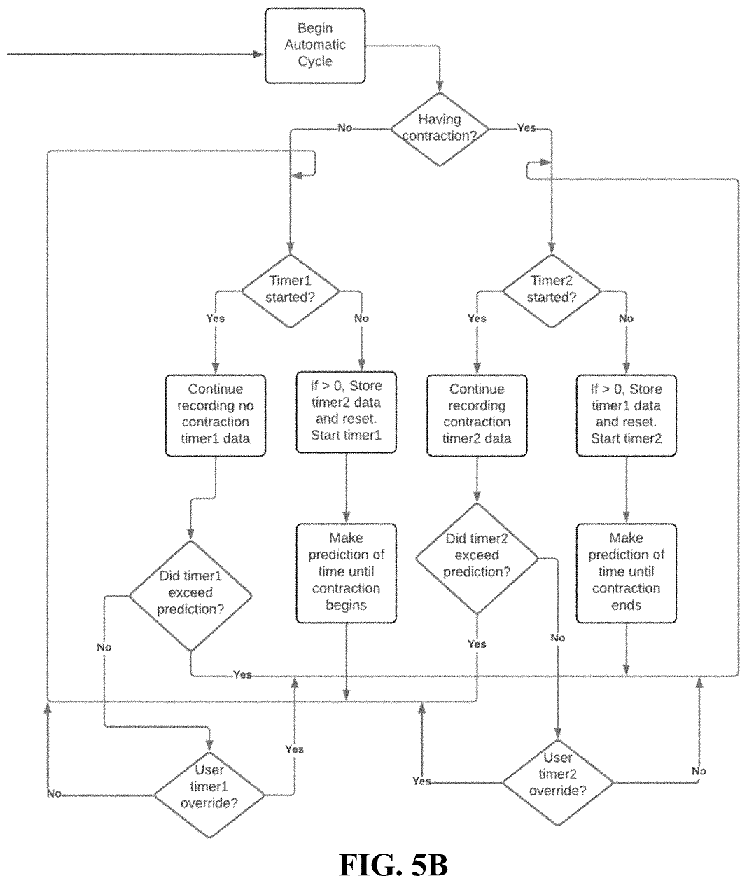

FIG. 5A and FIG. 5B are similar to the algorithms of FIG. 2A, except the system does not read patient data (such as patient uterine pressure rates) but instead relies on user input to make its calculations.

For example, in at least some embodiments utilizing an algorithm like the one shown in FIG. 5A and FIG. 5B, the user input is a normal contraction timer. When a contraction is detected the patient and/or their birthing partner can manually activate the timer. After enough data is received about the contraction times (including their frequency and durations) the algorithm can generate predictions of the amount of time until future contraction(s) begin(s) and end(s).

In some embodiments, the algorithm can predict up to two contractions into the future. In some embodiments, the algorithm can predict up to three contractions into the future. In some embodiments, the algorithm can predict up to four contractions into the future. In some embodiments, the algorithm can predict up to five contractions into the future. In some embodiments, the algorithm can predict up to six contractions into the future.

In some embodiments, the algorithm and application offer earlier detections of a contraction when compared to traditional methods. For example, doctors often use an average minimum value of 50 to detect when a contraction is in progress. In some embodiments, the algorithm can form this detection when the uterine pressure strength is as low as 18.

In some embodiments, the application and algorithm can predict the length of the patient's labor process. In some embodiments, the application and algorithm can predict estimated birth times.

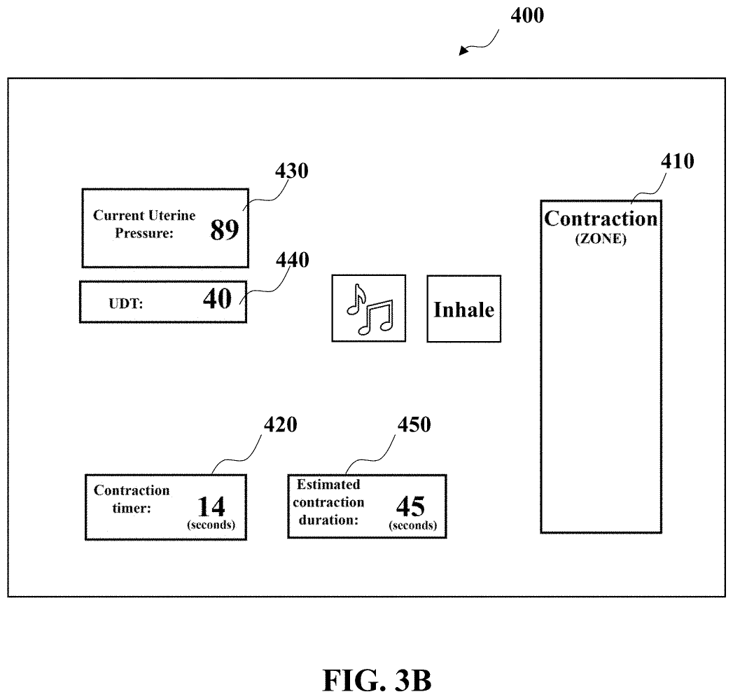

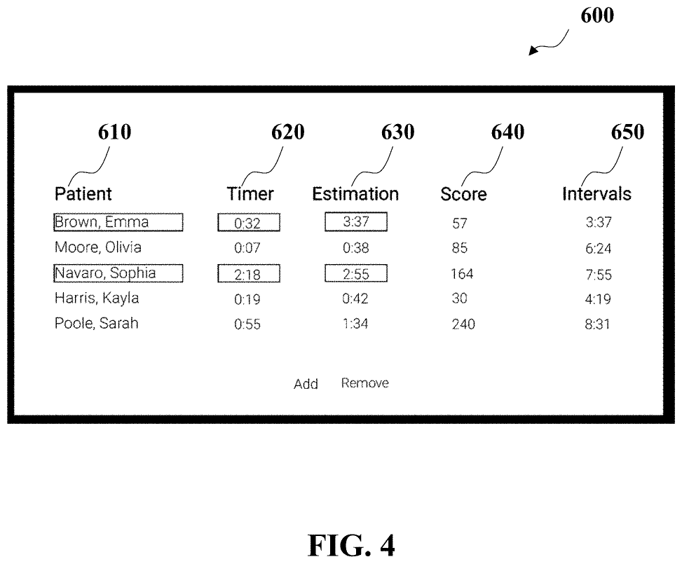

FIG. 3A provides an example graphical user interface 300 that can be displayed on a device or a separate display. In some embodiments, graphical user interface 300 is configured to be displayed to a patient. As depicted, graphical user interface 300 provides the current uterine pressure. In at least some embodiments, graphical user interface 300 can be configured based on a comfort threshold that the patient can select. In some embodiments, information on graphical user interface 300 is determined based on this selected threshold and the patient's current pressure level. In at least some embodiments, the comfort pressure level is determined, at least in part, by the patient. For example, in some embodiments, as labor progresses a given patient's pain threshold can increase.

In one example, a patient can set their initial pressure threshold to a lower number, such as 25 Montevideo units and under, as being tolerable. In some embodiments, they can change that tolerable value to a higher number, such as 40 Montevideo units and under, as more painful contractions occur. In some embodiments, the algorithm detects what these values are and computes moments when a painful pressure increase will occur based on the patient's current threshold.

In some embodiments, graphical user interface 300 can display Timer 320 that can show the time from the end of a previous contraction, and/or an estimated time until the start of a next contraction. In some embodiments, Timer 320 can be toggled on/off based on the user's preference.

In some embodiments, graphical user interface 300 shows Comfort Zone Panel 310 depicting the current state of the patient based on their uterine pressure. In some embodiments, the state of the Comfort Zone Panel 310 corresponds to a period in which the patient should not be having a contraction and should be experiencing a relatively low uterine pressure. In some embodiments, Comfort Zone Panel 310 changes color based on the patient's uterine pressure, which should relate to whether a patient is having and/or about to have a contraction.

In some embodiments, Comfort Zone Panel 310 is blue when a patient is between contractions and orange when a patient is experiencing a contraction.

In some embodiments, Montevideo score 370 is calculated and displayed on graphical user interface 300. In some embodiments, a patient can turn the Montevideo score 370 on and off depending on whether they want to see it or not. In some embodiments, the color of Comfort Zone Panel 310 can change based on the labor progression detected and/or a Montevideo unit score greater than or equal to a given number (such as 200). In some embodiments, the Montevideo score is displayed in Comfort Zone Panel 310.

In some embodiments, graphical user interface 300 includes a Current Uterine Pressure Panel 330 configured to show the current uterine pressure of a patient. In some embodiments, graphical user interface 300 includes a UDT (User Defined Threshold) Panel 340. In some embodiments, the patient can set a personal uterine pressure strength value, which corresponds to the user's pain tolerance, that activates whenever the monitored uterine pressure strength surpasses that value.

In some embodiments, graphical user interface 300 includes Estimated Time of Contraction Panel 350 configured to show the length of a previous contraction. In some embodiments, Estimated Time of Contraction Panel 350 is configured to show the estimated length of an upcoming contraction.

In some embodiments, an algorithm, such as the one shown in FIG. 2B, uses a patient's uterine pressure strength to identify if the patient is having a contraction or not. In some embodiments, the algorithm goes through the previously stored data which holds the times until a contraction occurred. Using pattern recognition along with artificial intelligence that grows accustomed to the user's contraction interval tendencies and real time occurrences, the algorithm can form a prediction. In some embodiments, if the predicted time is exceeded and a contraction does not occur, the algorithm and predicted time can automatically update.

In some embodiments, display unit 140 and/or device 150 can output motivational messages to the patient through graphical user interface 300 and/or graphical user interface 400. For example, in some embodiments when Comfort Zone Panel 310 is showing a low uterine pressure, graphical user interface 300 displays motivational and positive statements. In some embodiments, the motivational and positive statements appear in Comfort Zone Panel 310. In some embodiments, display unit 140 can be configured to display patient selected and implemented messages at specified moments of uterine pressure and consistency. In some embodiments, the system is configured to display these motivational messages when a contraction is occurring. In some embodiments, the system is configured to display these motivational messages when there is a low possibility of a contraction.

In some embodiments, graphical user interface 300 and/or graphical user interface 400 include panel(s) a patient can used to activate customizable features such as playing stored music. In some embodiments, graphical user interface 300 and/or graphical user interface 400 includes a panel that offers instructions to a patient such as breathing techniques. In some embodiments, graphical user interface 300 and/or graphical user interface 400 includes a single countdown of the estimated time of a current contraction or the estimated time until a new contraction.

FIG. 3B illustrates an example graphical user interface 400. Similar to FIG. 3A, graphical user interface 400 provides the current uterine pressure and threshold. In addition, graphical user interface 400 displays Timer 420 that can show the time for the end of a previous contraction, and/or an estimated time until the start of a next contraction. In some embodiments, Timer 420 can be toggled on/off based on the user's preference.

In some embodiments, Contraction Zone Panel 410 corresponds to the state of a patient based on their uterine pressure. In some embodiments, when a contraction is either on the way or occurring the patient experiences a relatively high uterine pressure.

In some embodiments, graphical user interface 400 includes Current Uterine Pressure Panel 430 configured to show the current uterine pressure of a patient. In some embodiments, graphical user interface 400 includes a UDT Panel 440

In some embodiments, graphical user interface 400 includes Estimated Time of Contraction Panel 450 configured to show the length of a previous contraction. In some embodiments, Estimated Time of Contraction Panel 450 is configured to show the length of a current contraction. In some embodiments, Estimated Time of Contraction Panel 450 is configured to show the estimated length of an upcoming contraction.

In some embodiments, the system can detect and/or predict when a minimal pressure change is going to occur while the patient is experiencing low and steady pressure to fortify optimal comfortability.

In some embodiments, specific colors are utilized throughout graphical user interfaces 300 and/or 400 to bring calm and soothing atmospheres. In some embodiments, the patient can preselect these colors.

In some embodiments, blue is used in moments when a contraction is not occurring or has just ended, orange is used when a contraction is occurring, and/or pink represents when the patient has surpassed a given Montevideo score for a given interval.

In some embodiments, during contractions, graphical user interface 400 can provide a button that can be selected to offer the patient “comforting stimuli” based on at least one of the five basic senses. In some embodiments, the button is Contraction Zone Panel 410. In some embodiments, the button is Comfort Zone Panel 310.

For example, in some embodiments, graphical user interface 300 and/or graphical user interface 400 can display selected and/or stored images and/or videos. In some embodiments, these images/videos can be preselected by a patient from their personal images such as family photographs and/or videos. In some embodiments, these images and/or videos can be randomly selected from a group of preselected images and/or videos. In some embodiments, the system can select images and/or videos based on patient data such as, but not limited to, an initial survey to determine images and/or videos that might be calming/comforting to the patient and/or data collected by the system on how the patient responded to previous images and/or videos. In some embodiments, a patient can set a particular image/video to display when a particular condition is reached, such as a patient's water breaking or the start or end of a contraction. In some embodiments, the condition is a predicted period of low uterine pressure levels, or a set amount of time before the algorithm predicts a contraction will occur.

In some embodiments, audio files such as, but not limited to, music, white noise, and/or recorded voices are played by the display. In some embodiments, these audio files can be preselected by a patient from their personal audio files such as favorite songs or prerecorded messages from family and friends. In some embodiments, these audio files can be randomly selected from a group of audio files. In some embodiments, the system can select audio files based on patient data such as, but not limited to, an initial survey to determine audio files that might be calming/comforting to the patient and/or data collected by the system on how the patient responded to previous audio files. In some embodiments, a patient can set a particular audio file to play when a particular condition is reached, such as a patient's water breaking or the start or end of a contraction. In some embodiments, the condition is a predicted period of low uterine pressure levels, or a set amount of time before the algorithm predicts a contraction will occur.

In some embodiments, scents can be selected and outputted by the system. In some embodiments, these scents can be preselected by a patient from their collection such as perfumes or essential oils. In some embodiments, these scents can be randomly selected from a group of scents. In some embodiments, the system can select scents based on patient data such as, but not limited to, an initial survey to determine scents that might be calming/comforting to the patient and/or data collected by the system on how the patient responded to previous scents. In some embodiments, a patient can set a particular scent to be released when a particular condition is reached, such as a patient's water breaking or the start or end of a contraction. In some embodiments, the condition is a predicted period of low uterine pressure levels, or a set amount of time before the algorithm predicts a contraction will occur.

In some embodiments, the system sends a notification to an individual, such as a birthing partner and/or healthcare provider, that a particular scent has been requested. In some embodiments, the system can predict when certain scent related items will be requested based on the progression of the labor and send a notification to an individual in advance.

In some embodiments, tastes can be selected and outputted by the system. In some embodiments, the taste desired is ice chips. In some embodiments, the system sends a notification to an individual, such as a birthing partner and/or healthcare provider, that a particular taste related item, such as ice chips, has been requested. In some embodiments, the system can predict when certain taste related items will be requested based on the progression of the labor and send a notification to an individual in advance.

In some embodiments, these tastes can be preselected by a patient from their collection. In some embodiments, these tastes can be randomly selected from a group of tastes. In some embodiments, the system can select tastes based on patient data such as, but not limited to, an initial survey to determine tastes that might be calming/comforting to the patient and/or data collected by the system on how the patient responded to previous tastes. In some embodiments, the patient selects the desired taste on the application. In some embodiments, a patient can set a particular taste to be released when a particular condition is reached, such as a patient's water breaking or the start or end of a contraction. In some embodiments, the condition is a predicted period of low uterine pressure levels, or a set amount of time before the algorithm predicts a contraction will occur.

For touch, in some embodiments, the application presents options to access the patient's own neurophysiological pathways to bring more comfort during labor. In some embodiments, the system sends a notification to an individual, such as a birthing partner and/or healthcare provider, that a particular stimulation is required such as, but not limited to, Merkel's Disk stimulation which can be activated via holding hands or squeezing. In some embodiments, the patient desires Pacinian corpuscles stimulation which can be activated via vibration. In some embodiments, the system can predict when stimulations will be requested based on the progression of the labor and send a notification to an individual in advance. In some embodiments, the patient selects the desired simulation on the application.

In some embodiments, the system can be configured to offer a selection that orchestrates a private social environment for the patient. For example, in some embodiments, messages filled with love and inspiration can appear on the graphical user interface from loved ones. In some embodiments, through authorized network connectivity, loved ones can communicate their messages and view a moderated version of the labor progress of the patient. In some embodiments, communication devices such as laptops, smartphones, desktop computers and/or tablets can be used to interface with the server.

In some embodiments, the application can take the place of a traditional birthing partner. In some embodiments, the application can alert preidentified individuals (such as family and/or friends) when a patient has reached milestones of the labor process.

In some embodiments, the application can be customized to a particular patient. For example, in some embodiments, a patient can load a profile picture to be associate with their profile, change the background color of the application, choose what colors relate to various labor conditions, and/or the like. In some embodiments, the application can require a password from the patient to access their custom settings and/or data.

In some embodiments, the application can have features that allow a patient and/or birthing partner to take a picture and/or video during the labor to record memorable moments such as the baby's birth. In some embodiments, the application utilizes a camera on the device the application is running on. In some embodiments, the device running the application is connected (either wirelessly or with a wire) to a separate camera that can be utilized.

In some embodiments, the application can offer pastime activities, such as games, puzzles, movies, and/or other types of entertainment. In some embodiments, the application can interact with other applications, such as video streaming services to provide entertainment.

In some embodiments, the application can be used prior to the patient's hospital visit, connecting the patient with associated labor coaches and/or doctors to help the patient properly prepare for their upcoming labor. In some embodiments, the application can alert the expecting mother when it is time for them to head to the hospital. In some embodiments, this is done based on the patient's contractions.

In some embodiments, the server can detect how long the patient is pushing for during the second stage of labor. In some embodiments, this information can be displayed through a graphical user interface. For example, in some embodiments, over a 10-minute interval the server can output to the patient an estimate of how long they were pushing. In some embodiments, it can indicate what their outputted strength was during moments of a contraction. In some embodiments, this information can be compared to the requirements and expectations of doctors and nurses, efficiently keeping the patient informed about their current state.

In some embodiments, the application can be configured to estimate how long the second stage of labor will last. Through numerous uses of the application, stored information can be utilized to show the average length of the second stage of labor.

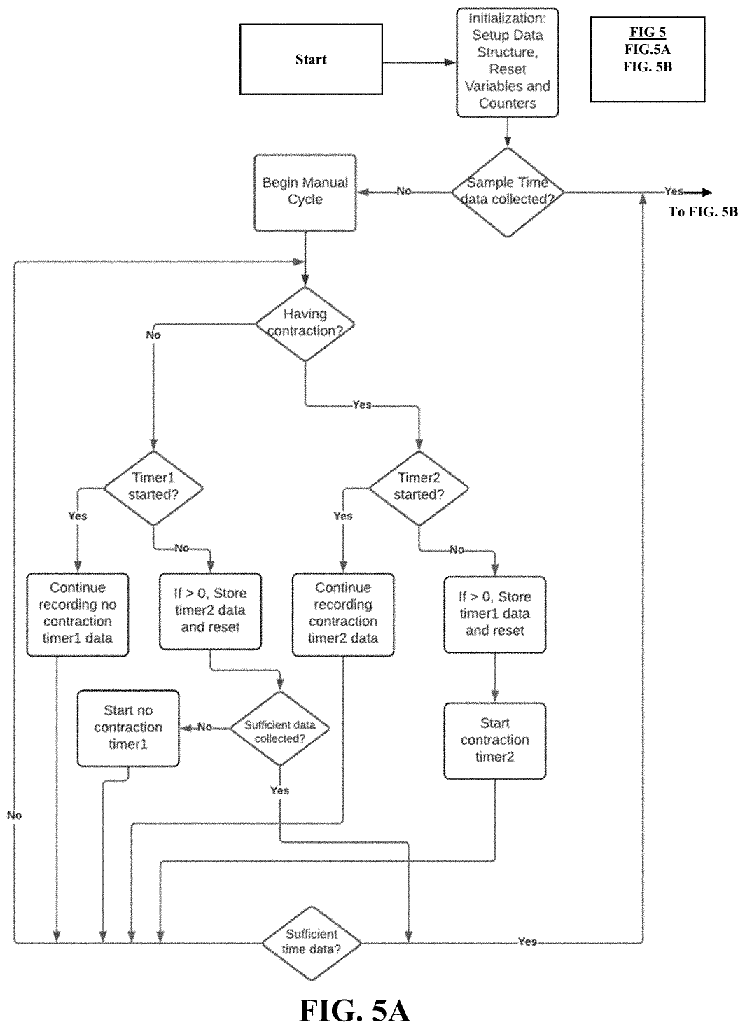

FIG. 4 is an example of a graphical user interface for displaying user related uterine information for multiple patients. Practitioner Hub 600 can be utilized by medical service providers, such as doctors and nurses, to monitor multiple patients at a time. In some embodiments, Practitioner Hub 600 can include Patient ID Information 610, Contraction Timer Information 620, Contraction Estimator Information 630, Score Information 640, and/or Interval Information 650 for each patient.

In some embodiments, Patient ID Information 610 includes the patient's name and/or room number.

In some embodiments, Practitioner Hub 600 can show each patients' current contraction time and/or the time to their next contraction. In some embodiments, Practitioner Hub 600 includes an interval timer Interval Information 650 at which resets itself and the labor score at Score Information 640 at regular intervals (such as every ten minutes).

In some embodiments, Practitioner Hub 600 can present a particular patient's information in a particular color depending on what part of labor they are currently in. For example, in some embodiments, if the patient is experiencing a contraction their information can be orange. In some embodiments, if the patient is not experiencing a contraction their information can be blue. In some embodiments, if the patient has an interval score above a given number (such as 200) their information can be pink. In some embodiments, the patient can change these colors.

In some embodiments, voice messages can be sent through Practitioner Hub 600 to a specified patient and vice versa. In some embodiments, nurses and doctors can use Practitioner Hub 600 to record the patient's dilation.

In some embodiments, the application can be a mobile-based, cloud-based, server based and/or online-based application. In some embodiments, the devices can be a smartphone, tablet, laptop, smartwatch, or personal computer. In some embodiments, the application can be hosted by a commercially available platform. In some embodiments, the application can be embodied in a non-transitory computer-readable storage medium.

Computer readable medium having program code recorded thereon for execution on a computer of the methods are disclosed above.

In some embodiments, a computer program can input uterine strength data and patient information and output a graphical user interface with information optimized at making their childbirth more comfortable.

Throughout the description, specific details are set forth to provide a more thorough understanding of the invention. However, the disclosed methods and systems can be practiced without these particulars. In other instances, well-known elements have not been shown or described in detail to avoid unnecessarily obscuring the invention. Accordingly, the specification and drawings are to be regarded in an illustrative, rather than a restrictive sense.

Unless the context clearly requires otherwise, throughout the description and the claims:

“comprise”, “comprising”, and the like are to be construed in an inclusive sense, as opposed to an exclusive or exhaustive sense; that is to say, in the sense of “including, but not limited to”;

“connected”, “coupled”, or variants thereof, mean connection or coupling, either direct or indirect, permanent, or non-permanent, between two or more elements; the coupling or connection between the elements can be physical, logical, or a combination thereof;

“herein”, “above”, “below”, and words of similar import, when used to describe this specification, shall refer to this specification as a whole, and not to any particular portions of this specification;

“or”, in reference to a list of two or more items, covers all of the following interpretations of the word: any of the items in the list, all of the items in the list, and any combination of the items in the list;

the singular forms “a”, “an”, and “the” also include the meaning of any appropriate plural forms.

Specific examples of systems, methods and apparatuses have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

What is claimed is:

1. A system for making labor more comfortable and/or efficient comprising:
(a) a monitor configured to measure patient data;
(b) a computer; and
(c) a display.

2. The system of claim 1 wherein said system further comprising:
(d) a server.

3. The system of claim 1 wherein said monitor monitors a uterine pressure strength of a patient.

4. The system of claim 1 wherein said monitor is a fetal monitor.

5. The system of claim 1 wherein said monitor is a maternal monitor.

6. The system of claim 1 wherein said patient data is analyzed by said computer to determine when a patient is having a contraction.

7. The system of claim 1 wherein said patient data is analyzed by said computer to predict when a patient will have a contraction.

8. The system of claim 1 wherein said display is configured to display a graphical user interface.

9. The system of claim 8 wherein said graphical user interface includes a Comfort Panel.

10. The system of claim 9 wherein said Comfort Panel changes between colors whether a patient is experiencing a contraction.

11. The system of claim 8 wherein said graphical user interface includes a Montevideo score.

12. The system of claim 11 wherein said Montevideo score is updated at regular intervals.

13. The system of claim 1 wherein said monitor is an intrauterine pressure catheter sensor.

14. The system of claim 1 wherein said system can output a motivational messages on said display.

15. The system of claim 1 wherein said system is configured to offer a comforting stimulus.

16. The system of claim 15 wherein said comforting stimulus is an audio sound.

17. The system of claim 15 wherein said comforting stimulus is an image or video.

18. The system of claim 15 wherein said comforting stimulus is a scent.

19. The system of claim 1 wherein a patient inputs data about their contractions and said data is analyzed by said computer to predict when said patient will have a future contraction.

20. The system of claim 1 wherein said system if part of a plurality of systems connected to a hub configured to allow a medical service provider to monitor multiple patients.
